(19) United States
(12) Patent Application Publication
(10) Pub. No.: US 2026/0350893 A1
(43) Pub. Date: Nov. 5, 2026

(54) Streaming Read-Out Data Acquisition and Scalable Distributed Analysis Framework for Nuclear Medical Imaging

(71) Applicant: JEFFERSON SCIENCE ASSOCIATES, LLC, Newport News, VA (US)

(72) Inventors: Cameron Clarke, Newport News, VA (US); Vardan Gyurjyan, Seaford, VA (US); John McKisson, Yorktown, VA (US); Benjamin Raydo, Williamsburg, VA (US); Andrew Weisenberger, Springfield, VA (US)

(73) Assignee: JEFFERSON SCIENCE ASSOCIATES, LLC, Newport News, VA (US)

(21) Appl. No.: 19/643,469

(22) Filed: Jun. 12, 2026

Related U.S. Application Data
(60) Provisional application No. 63/785,910, filed on Apr. 9, 2025.

(51) Int. Cl. G06T 12/30; A61B 6/03; H04L 47/125; H04L 47/19

(52) U.S. Cl. CPC G06T 12/30; A61B 6/037; H04L 47/125; H04L 47/19; G06T 2210/41

(57) ABSTRACT

A method and system for processing streaming nuclear imaging data in a distributed system which reconstructs time-synchronized events over load-balanced and multi-path transport, while preserving consistency with an in-band control-state model, and maintaining continuous operation across state transitions. A continuous stream of digitized event data is generated at multiple detector arrays during the imaging process. The event data is segmented into transport units and distributed in a load balanced manner across a local computing resource, a remote computing cluster, or multiple high performance computing cluster nodes depending on resource availability and other pre-determined criteria. When applied to positron emission tomography, a downstream software solution simultaneously processes the event data and completes the necessary geometry analysis that was previously done in front-end hardware devices. The method and system preserves determinism despite multi-path transport, heterogeneous compute nodes, and network latency variations.

This application claims priority from U.S. Provisional Patent Application No. 63/785,910 filed on Apr. 9, 2025.

The United States Government may have certain rights to this invention under Management and Operating Contract No. DE-AC05-06OR23177 from the Department of Energy.

### FIELD OF THE INVENTION

The present invention relates to processing nuclear medicine data from an imaging examination, and more specifically to processing nuclear imaging data using distributed computing systems.

### BACKGROUND

Nuclear medicine diagnostic procedures for patients involve the imaging of the distribution of injected biochemical compounds chemically linked with a radioactive nuclide. Radioisotope imaging detectors may be used to image the location and distribution of the radiopharmaceutical after the tracer has been injected into a patient. The temporal and spatial way a radiopharmaceutical compound is distributed in the tissue of the patient is dependent on the metabolism and biological function of that compound. Thus, by imaging the biodistribution of the radiopharmaceutical, information about the tissue uptake rate and distribution is obtained. Positron Emission Tomography (PET) is a nuclear medicine imaging modality which uses positron emitting radiopharmaceuticals.

With PET three-dimension information of the biodistribution of the radiopharmaceutical that was injected in the patient may be obtained. In PET, the radioisotope decays by a physical process called positron emission. The positron that is emitted in the nuclear decay is an example of a class of particles known as anti-matter. The positron has the same mass of an electron however it has the opposite charge. When anti-matter and matter interact, the encounter results in the annihilation of the two with the release of energy. When a positron and an electron interact, they annihilate resulting in the complete conversion of the electron and positron into a pair of 511 keV photons (often called gamma-rays) traveling at nearly 180° opposite directions from the annihilation point. The high energy gamma-ray easily escapes out of the patient's body. Typically, gamma-rays can be detected by using dense transparent scintillation crystals. It is the coincident detection of the two oppositely directed 511 keV gamma-rays that is used to compute a tomographic back projection and thus reconstruct a three-dimensional image of the biodistribution of the emitting source.

A typical clinical PET system is made of a stationary ring of several detector modules made of crystal scintillators which generate fast scintillation photon pulses that are read out by individual arrays of photomultiplier tubes or other scintillation photon detectors such as silicon photomultipliers resulting in many channels transporting electrical signals digitizing electronics. The patient is on a translating bed which moves the patient through the center of the detector ring such that the desired length of the region of interest of the body is scanned by the PET ring during the procedure. The many analog electrical signals from the detectors which pass a per-detector and per-gamma ray detection level 1 trigger condition are then electronically digitized allowing their computer analysis and storage.

Conventional PET scanners often employ field-programmable gate array (FPGA) data acquisition systems. These scanners use front-end electronics, i.e., hardware located proximate to the detector arrays, to identify imaging data of interest. In such systems, a level-2 trigger system is employed to determine coincidence and anti-coincidence of detection events. Any data not passing the level-2 trigger decision is discarded and not passed further into the system or stored for future evaluation.

The operational efficiency of such a conventional system may be increased through the addition of detectors in order to, inter alia, obtain greater image resolution and increase sensitivity. However, an increase in the number of detectors results in an increase in the latency, i.e. rate-dependent pile-up induced paralyzation, of the system. Latency is one of the primary contributors to detector “dead time”, the period during which the detector is essentially blind to imaging data while the trigger system is busy analyzing prior events.

One potential solution, which is currently being explored in the field, to this problem is the use of streaming data acquisition. Instead of relying on a level-2 trigger system, all imaging data is passed into the system with minimal initial filtration or processing. Data analytics are deferred to software processing operations further downstream rather than in the front-end hardware of the system. As a result, no event data is lost or discarded during the imaging process. One problem with this approach, at least when applied to total body PET scanning, is that the continuous data from a large number of detector streams that needs to be processed in parallel will saturate and overwhelm the networking and computational processing capabilities of standard computing systems.

This is pertinent, in part, because existing commercial PET systems do not have a long enough axial bore to image the entire body of a patient in one pass. Aside from the fact that it would require considerable financial resources to construct a system that incorporates the necessary number of detectors for whole body imaging, it is extraordinarily challenging to build an integrated DAQ system that processes the required triggers. Or, in the case of a streaming system relying upon triggerless acquisition of imaging data, it is a challenge to assemble a distributed processing system that can handle the quantity of detector streams and potential coincident geometry pairs from such a large number of detectors. By way of example, a total body PET system could generate in excess of 100 Gbps of continuous streaming data. The foregoing practical limitations essentially prevent any scaling up of a total body PET scanner beyond a particular number of data streams.

Accordingly, there is a need for a scalable data acquisition and processing system for a total body PET device that addresses the obstacles that would normally hinder the use of a large number of detectors generating independent data streams, in order to, among other things, increase the sensitivity of the scan, improve the quality of the image, decrease the dose of the administered radiopharmaceutical, and/or improve image acquisition time.

### SUMMARY OF THE INVENTION

A method and system for processing streaming nuclear imaging data in a distributed system which reconstructs time-synchronized events over load-balanced and multi-path transport, while preserving strict consistency with an in-band control-state model, and maintaining continuous operation across state transitions. This method differs from conventional stream processing by providing physics-level event reconstruction guarantees over distributed, load-balanced, and non-deterministic transport environments.

When applied to total-body positron emission tomography (PET), detector data is generated by multiple streaming readout field-programmable gate arrays, fragmented into event pieces, and transported through a high-throughput network. Event pieces are distributed in load-balanced manner across system processing endpoints, based on (i) event identifier, (ii) a time window, and/or (iii) adaptive load conditions of the system. The event data can be, and consistently is, intentionally reordered during transport and is realigned downstream. Downstream processing is performed by microservice framework actors distributed across compute nodes. Unlike a conventional PET system, the downstream software solution, capable of receiving the stream packaged data units from the single detector units simultaneously processes the event data and completes the necessary geometry analysis that was traditionally executed in front-end hardware devices

The disclosed method and system simplifies the entire PET system readout into a software defined package of independent modular detectors with intrinsically parallelized real-time analysis using a time-interleaving method. The streaming readout using highspeed network to distribute time-interleaved data for processing substantially reduces or eliminates PET event processing bandwidth bottleneck and prevents lost counts and system inefficiencies.

### BRIEF DESCRIPTION OF THE DRAWINGS(S)

FIG. 1 depicts the elements of an illustrative embodiment of the method and system and an exemplary workflow within that embodiment.

### DETAILED DESCRIPTION

The present disclosure teaches a method and system of data acquisition and processing for certain types of medical nuclear imaging, e.g., total-body PET, which simplifies the entire total-body-PET system processing and readout.

The method and system uses a highspeed network to distribute time-interleaved information packets for processing. This architecture and process provides numerous benefits, such as the elimination of PET event processing bottleneck, and prevention of lost counts and overall system inefficiencies. It enables a uniform PET scanner building strategy with all software defined PET system geometry and time-interleaving computation/data processing methodology handled by an automated orchestrator within an actor-based microservice framework and provides a simple, user friendly, and standardized data input, analysis prescription, and data output interface for all stages of detector development, prototyping, and system implementation for total-body-PET scanner development-as well as for work outside of the specific field of PET after suitable reconfiguration of the data streaming read-out and processing architecture herein described.

This parallelizability is also positioned to take advantage of additional optimization systems to further improve efficiencies and performance. For instance, the JLAB Integrated Research Infrastructure Across Facilities (JIRIAF) system at the Thomas Jefferson National Accelerator Facility (Jefferson Lab) enables robustly and fairly sharing high performance computing (HPC) resources within a HPC cluster if the resources are needed promptly or the need is too great for standard resource reservation allocation on a single HPC node. In addition, the Jefferson Lab ESnet-JLab FPGA Accelerated Transport (EJFAT) Load Balancer system can further federate the HPC resources across multiple HPC clusters within the ESNet system, if multiple clusters need to be engaged simultaneously, while maintaining the ability to interconnect analysis processes with shared data.

The benefit of implementing this method and system for PET research is to provide a standard full analysis framework that is user friendly and interchangeable for various users, tolerates changes in analysis topologies, supports arbitrary data input and output for detector prototyping and bench-top tests, all while maintaining the same interface as is to be used in the final clinical system (i.e. the clinical system software support prototyping, benchmarking, and simulated data inputs, not just the final system topology).

Further, the benefit of implementing this framework for PET clinical systems is that the resulting data acquisition and analysis systems can interface both with local resources for guaranteed analysis work on-disk and GPU, while also simultaneously interfacing the raw or processed data streams to distributed (local or multi-node) high performance computing resources for real-time processing, thus reducing the need for advanced data processing hardware on site for real-time processing, thereby reducing costs for end users already burdened by the expensive detector arrays of total-body-PET, and enabling the use of available remote high computing power for rapid Monte Carlo enhanced, GPU enabled iterative recon, and beyond.

Referring now to the use of the disclosed system and method specifically in conjunction with positron emission tomography (PET) medical imaging, extraordinarily large volumes of data are generated during the PET scanning process. Digital streaming readout capable radiation detector modules generate continuous streams of data using free-running analog-to-digital conversion front-end electronics with triggerless acquisition. The digitized data from these electronics are assembled into Structured Data Frames. Structured Data Frame(s) contain PET positron annihilation gamma ray or other relevant physics observable events'arrival time, energy, position information or other such metrics digitized with sub-segmented channels in front-end electronics and, further, contain the location information and meta-data associated with the detector module placement of a single detector, array of detectors, or clinical scanner.

The Structured Data Frames from each PET detector module's front-end electronics are contained in TCP/UDP/IP based packets. These networking capable packets are referred to as structured streaming event packets or SSEPs. TCP/UDP/IP or other network methodologies are implemented for transmission of the SSEPs in modular detector front-end or aggregation systems and are implemented for reception within data processing components (actors) integrated within an actor-based microservice framework, such as the Jefferson Lab Environment for Real-time Streaming, Acquisition and Processing (ERSAP) system.

The microservice framework actors perform time interleaving, essentially grouping data fragments by desired event time or window of time, with the event buffers of SSEPs and distribute relevant SSEP buffers according to their arrival time information to one or plural number of computation threads containing inside a CPU or a GPU, or inside many of these CPU/GPU units distributed within a local computing resource, to a remote computing cluster, or to multiple high performance computing cluster nodes depending on resource availability and the need identified by the microservice framework orchestrator process. The use of a microservice framework to perform time-interleaving network distribution and horizontal multi-node and vertical multi-threaded parallelized processing of read out data streams enables arbitrarily large and complicated networks of analysis actors as long as sufficient parallel computing resources are available.

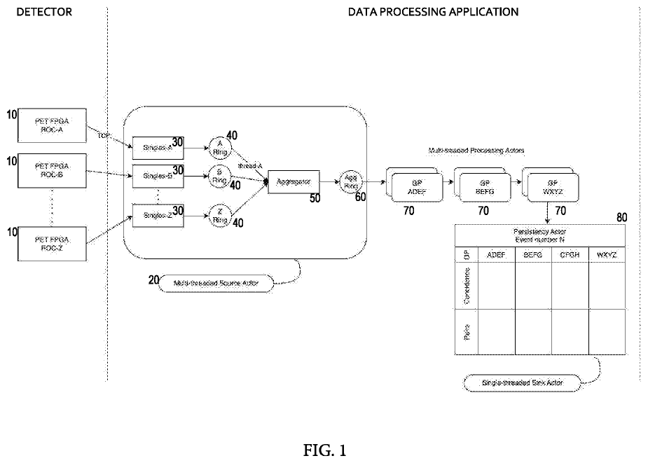

The foregoing method and system is more fully illustrated in FIG. 1 which provides an illustration of data stream processing and analysis architecture. Referring now to that diagram, a data stream of observable event data is collected during PET imaging by, for example, a group of detector arrays (10). The event data would include the time, energy, and location of incident events. A streaming readout (SRO) field-programmable gate array (FPGA) in each detector array (10) produces an Internet capable continuous data stream of the event data for that array (10). The continuous data stream is transmitted, as discussed in further detail herein, through standard Internet protocols from the detector array(s) (10) through the processing system. In this embodiment, the data stream is transmitted using Transmission Control Protocol (TCP), although other such Internet communication protocols may be selected.

The novel data processing begins with the production of event fragment data at the SRO FPGAs (10). Each fragment is associated with an event number and/or a high-resolution timestamp from a synchronized timing system. The data stream of event fragments representing single events data is passed to a multi-threaded source actor (20) within the microservice framework. The microservice framework actor (20) performs time interleaving with the event buffers of SSEPs and distributes relevant SSEP buffers according to their arrival time information to one or plural number of computation threads containing inside a CPU or a GPU, or inside many of these CPU/GPU units distributed within a local computing resource, to a remote computing cluster, or to multiple high performance computing cluster nodes depending on resource availability and the need identified by the orchestrator process. The use of microservice framework actors to perform time-interleaving network distribution and horizontal multi-node and vertical multi-threaded parallelized processing of read out data streams during various phases of data processing enables arbitrarily large and complicated networks of analysis actors as long as sufficient parallel computing resources are made available to the microservice framework. In a preferred embodiment, system resource configuration and availability is determined by the user(s) and communicated to the system through the use of input files at run-time.

Initial threshold processing, such as the implementation of PET calibration procedures or the application of such other correction factors that are empirically determined for the system, is performed on the single events raw data (30). The minimally processed singles data can then be passed to one or more pre-allocated blocks of memory (40) for temporary storage. All of the processed and stored singles data is subsequently aggregated (50) and then passed to another pre-allocated block of memory (60) for temporary storage. The data is then ready for sequential geometry processing in order to determine coincident events or to perform another such assessment.

Event fragments are segmented and transmitted through the network such that a single event may correspond to multiple packets and packet delivery to subsequent processors may occur out-of-order. Fragments are distributed in load-balanced manner across system processing endpoints, based on (i) event identifier, (ii) a time window, and/or (iii) adaptive load conditions of the system. Microservice framework actors receive these fragments and maintain local or distributed event assembly buffers, which index fragments by event number and/or timestamp. These actors track completeness across multiple sources and preserve state for partially assembled events.

A deterministic event alignment procedure is performed by multi-threaded processing actors. This procedure includes grouping fragments from varying detectors that belong to the same event, is executed enforcing ordering constraints based on event number or time and applying policies for missing or delayed fragments (e.g., timeout completion or partial reconstruction).

PET coincident or other relevant higher level physics analysis events can be evaluated and determined through all geometrically relevant single events by sharing the framework orchestrator and connecting the parallelizable actor network sequentially using antecedent system output as inputs to subsequent geometric coincidence or other higher level physics event analysis actors. Each geometry processing actor (70) queries the full data set for data which is relevant to the particular detector geometry the actor (70) is processing. The geometrically relevant PET single events determined by physics and geometry criteria for inclusion in geometric coincidence analysis actors or other higher level physics event selection definitions are preselected and software defined.

Complete physics list-mode events are reconstructed once completion conditions are satisfied. Reconstructed events are propagated through distributed network pipelines, ensuring that all actors observe consistent and deterministic event sequence. Optional feedback can be provided within the system to adjust load balancing, buffering thresholds, and flow control parameters. Finally, data is transported to long term storage (80) for future analysis or review.

The microservice framework implementation provides a standard prescription of acceptable data formats and analysis limitations that permits a range of suitable data input and analysis output formats for interfacing with the network of data streams and analysis processes. This permits the orchestrator to determine the parallelized distribution of all data streams and analysis actors across available computing resources at run-time, alleviating the user of all design considerations for distributed data handling and processing, providing a standard data interface for arbitrary detector or simulation inputs, analysis methodologies, and output for PET reconstruction or other relevant detector configurations or physics process analyses.

The use of the distributed computing systems described herein in enables real-time collection and processing of very high singles event rates and coincidence-identified event rates resulting from the high data throughput (in a preferred embodiment, sustained throughput exceeds 100+Gbps across distributed nodes) and precise timing resolution from state-of-the-art detector systems without the need to configure or change coincidence processing in electronics hardware or firmware, resulting in PET imaging systems with arbitrary choices of geometrical configurations, quantities of detectors, types of detectors, and activity rates, all limited only by the chosen detectors'readout deadtime and intrinsic coincidence timing resolutions. Moreover, the distributed nature of the system provides elasticity allowing an increase or decrease in computing and network resource use and allocation as required at any particular time.

The method and system set forth herein may be used for:
(1) pre-clinical applications involving animal models, where the higher isotope activity rates enable faster imaging to minimize the need for motion control or maximize the impact of time dependent sequences for functional imaging or multi-modal imaging purposes;
(2) clinical applications involving MRI system PET imaging inserts or PET/MRI multi-modal imaging systems, where the minimized data acquisition electronics handling singles processing only from arbitrarily small state-of-the-art detector front-end designs with arbitrarily distant network-connected streamed data coincidence processing both enable magnetic field-insensitive and safe to use PET imaging systems;
(3) total-body-PET clinical applications, as the system enables data acquisition and processing of the large quantities and unique geometrical configurations of detectors used to provide high sensitivity to events generated at any point within the human body, covering a very large geometrical area with a large quantity of detectors and detector channels;
(4) rapid throughput clinical imaging applications with standard or total-body-PET, as the system enables data acquisition and processing of the large quantities of detected singles and coincidence-identified events generated with increased applied radioisotope activity or increased sensitivity from total-body-PET;
(5) high time resolution clinical imaging applications with standard or total-body-PET enabling time-resolved functional imaging applications, multi-phase or multi-radiopharmaceutical and multi-functional imaging within the same patient in a timescale similar to standard PET imaging;
(6) extremely low-dose clinical imaging applications with standard or total-body-PET but using the same acquisition time as standard PET imaging with lower administered radiopharmaceutical activity rates;
(7) basic and applied PET detector and system research purposes, enabling easily constructed prototype systems, easier modifications and permutations for system performance research or clinical trial configuration changes, and lower cost and technical barriers to access state-of-the-art data acquisition and analysis systems;
(8) Time-of-Flight-PET clinical imaging applications, where the software implementation of time-of-flight geometric coincidence conditions bypass any need to implement coincidence analysis in electronics hardware or firmware, enabling real-time imaging with arbitrarily high timing resolution state-of-the-art detector systems and fully modularized and easily modified geometry configurations thereof;
(9) PET insert improvements to standard PET clinical imaging applications, where novel detector inserts augmenting the coverage of standard PET scanners may improve imaging coverage and performance of distal regions such as the head or extremities during standard thoracic scanning without the need to construct entire full rings or an entire new total-body-PET scanner; and/or,
(10) X-ray linac and hadron or ion beam radiotherapy clinical imaging, quality assurance, and diagnostic applications, including in-beam-PET for radiopharmaceutical imaging, prompt-PET imaging, and prompt-or delayed-activated-secondaries-PET imaging, enabling image guidance for treatment planning and patient positioning, adaptive treatment planning through image guidance or functional imaging, and functional tumor diagnosis through PET perfusion and hypoxia monitoring.

The description of the present invention has been presented for purposes of illustration and description, but is not intended to be exhaustive or limited to the invention in the form disclosed. Modifications and variations may be apparent to those of ordinary skill in the art without departing from the scope and spirit of the invention.

1. A method for processing streaming data in a distributed system during positron emission tomography comprising:
providing independent continuous streams of digitized event data from a plurality of hardware devices concerning positron emission tomography imaging events; said event data comprising event fragments with each said event fragment having a unique identifier;
packaging said independent continuous streams of event data in structured streaming packets wherein a single imaging event may correspond to one or more structured streaming packets;
transporting and delivering said structured streaming packets in a load-balanced manner to a plurality of event assembly buffers based upon the event data and the unique identifier within said packets;
maintaining the plurality of local or distributed event assembly buffers which index said event fragments, track event data completeness across multiple sources and preserve state for partially assembled events;
further distributing said structured streaming packets in load-balanced manner across processing endpoints based upon the event data and the unique identifier within said packets; said processing endpoints comprising a local computing resource, a remote computing cluster, or a multiple high performance computing cluster nodes depending on networking and computational resource availability or other pre-determined criteria;
performing horizontal multi-node and vertical multi-threaded parallelized processing of the event data in said streaming packets in order to execute a deterministic event alignment procedure on said data; and,
reconstructing imaging events from said aligned event data once pre-determined completion conditions are satisfied.

2. The method of claim 1 wherein said unique identifier is a high-resolution timestamp from a synchronized timing system.

3. The method of claim 1 wherein transporting and delivering said structured streaming packets does not occur in a chronological order based upon the timestamp of each said event fragments and, therefore, streaming packet delivery within the system may occur out of chronological order.

4. The method of claim 1 wherein said event fragments further comprise data concerning observable events generated during the PET imaging process.

5. The method of claim 4 wherein said event fragments comprise positron emission tomography (PET) positron annihilation gamma ray arrival time, energy, and position information.

6. The method of claim 5 wherein said transporting and delivering packets across processing endpoints is based upon one or more of arrival time, energy, and position information.

7. The method of claim 1 wherein said structured streaming packets are TCP/UDP/IP based packets.

8. The method of claim 7 wherein said structured streaming packets are TCP based packets.

9. The method of claim 1 wherein maintaining local or distributed event assembly buffers further comprises indexing said event fragments by timestamp.

10. The method of claim 1 wherein said deterministic event alignment comprising one or more of the following activities: grouping fragments belonging to the same event, enforcing ordering constraints based on event number or time, and applying policies for missing or delayed event fragments.

11. The method of claim 1 further comprising adjusting certain operational parameters of the data processing method during operation in order to improve efficiency or address networking or computational deficiencies.

12. The method of claim 11 wherein adjusting certain operation parameters further comprises adjusting the load balancing, buffering thresholds, or flow control parameters of the system.

13. The method of claim 11 wherein adjusting certain operation parameters comprises using software to allocate, reallocate, and coordinate the use of available networking and distributed computational resources in order to achieve the desired positron emission tomography operating parameters.

14. A system for processing streaming data in a distributed system during positron emission tomography comprising:
at least one front end detector module;
a microservices framework coupled to said at least one front-end detector module; said framework comprised of a plurality of hierarchical actors residing on computational hardware in communication with said microservices framework;
wherein said detector modules are configured to generate structured streaming event packets containing event fragment data through the process of obtaining information about single observable events during the positron emission tomography, adding a time designator to said information, and incorporating said information and time designator in TCP/UDP/IP based event packets; said detector modules being further configured to transmit the structured streaming event packets containing event fragment data to computational hardware through the microservices framework;
wherein the microservices framework deploys one or more system actors operating on said computational hardware to accomplish the following tasks:
processing the event fragment data by a plurality of singles processor actors to prepare time-window interleaved event structures;
building and maintaining a plurality of event buffers to temporarily store the time-window interleaved event structures according to the information and time designator within each respective event structure;
aggregating the time-window interleaved event structures by at least one aggregation actor;
transmitting said time-window interleaved event structures sequentially through a series of multi-threaded geometry processors, each of which relates to a specific grouping of detector geometry;
conducting sequential horizontal multi-node and vertical multi-threaded parallelized geometry processing of said time-window interleaved event structures in order to determine coincident events;
reconstructing positron emission tomography full events and using said reconstruction to form an image; and,
storing all or a portion of said time-window interleaved event structures in long-term memory;
and further, wherein said microservices framework coordinates the operation of the system and the transmission of data therein and, further, determines and allocates the available system resources in relation to the resources required at any given time to perform the tasks.
